(19) United States
(12) Patent Application Publication
(10) Pub. No.: US 2015/0238445 A1
(43) Pub. Date: Aug. 27, 2015

(54) COMPOSITION USING METFORMIN FOR PREVENTING OR TREATING IMMUNE DISEASES INCLUDING LUPUS

(71) Applicant: CATHOLIC UNIVERSITY INDUSTRY ACADEMIC COOPERATION FOUNDATION, Seoul (KR)

(72) Inventors: Mi-La Cho, Seoul (KR); Seon-Yeong Lee, Gyeonggi-do (KR); Min-Jung Park, Incheon (KR); Jae-Seon Lee, Gyeonggi-do (KR); Eun-Ji Yang, Seoul (KR); Eun-Kyung Kim, Seoul (KR); Jun-Geol Ryu, Gangwon-do (KR); Hye-Jin Son, Seoul (KR)

(21) Appl. No.: 14/428,920

(22) PCT Filed: Sep. 6, 2013
(86) PCT No.: PCT/KR2013/008083

§ 371 Date: Mar. 17, 2015

(30) Foreign Application Priority Data
Sep. 17, 2012 (KR) 10-2012-0102887

(51) Int. Cl. A61K 31/155

(52) U.S. Cl. CPC A61K 31/155

(57) ABSTRACT

The present invention relates to a composition for preventing or treating immune diseases through the suppression of B cell activity induced by metformin. More particularly, the present invention relates to a composition comprising a metformin compound or a pharmaceutically acceptable salt thereof as an active ingredient for preventing or treating immune diseases, wherein the composition is characterized by suppression or reduction of B cell activity which is a cause of disease. The present invention can be valuable in the use thereof for various autoimmune diseases as an immunosuppressant which can prevent or treat immune diseases by suppressing or reducing B cell activity and Th17 cell activity, which are causes of disease, or by promoting or increasing regulatory T cell activity.

### TECHNICAL FIELD

The present invention relates to a composition for the prevention or treatment of immune disease through the suppression of B cell activity induced by metformin, more precisely a composition comprising a metformin compound or a pharmaceutically acceptable salt thereof as an active ingredient for the prevention or treatment of immune disease, wherein the composition is characterized by suppressing or reducing the activity of a pathogenic B cell.

### BACKGROUND ART

Immune response is a kind of self-defense system against an antigen that invades or is inserted into the body from outside, which is divided into the primary immune response and the secondary immune response. The primary immune response is induced mainly by lymphocytes. Lymphocytes are generated in bone marrow and then are circulated through blood to lymphatic tissue, lymph node, spleen, and tonsil to attack the invaded antigen. In the meantime, the secondary immune response is mainly induced by B cells and T cells. When an antigen invades into our body, B cells are fast proliferated to produce an antibody against the antigen and the produced antibody performs humoral immunity while being circulated through body fluid. T cells are formed in thymus and then migrated to lymphatic tissue to perform cell-mediated immunity to attack the antigen directly.

The most important factor in the secondary immune response in which B cells and T cells are involved is to respond only to the non-self antigen without responding to the self antigen. Leaving the self antigen untouched is called immune tolerance. When there is a problem in immune tolerance, the self antigen itself can be a target of the immune system, that is the self antigen is attacked, resulting in autoimmune disease. The representative autoimmune diseases are type I diabetes, rheumatoid arthritis, Hashimoto's thyroiditis, and multiple sclerosis, etc.

To treat such autoimmune disease, such operations as therapeutic plasma exchange wherein plasma of an autoimmune disease patient is eliminated and then normal plasma is transplanted in the patient, selective removal wherein IgG antibody alone is eliminated from patient plasma by using protein-A and the remaining plasma is injected back to the patient, and autoantibody removal wherein a specific autoantibody is eliminated from patient plasma using an autoantigen protein and then the remaining plasma is injected back to the patient are performed or medicinal therapy such as the administration of steroids or other immunosuppressants is performed. However, the above operations have problems of complicated processes and side effects. There is also a difficulty in performing the medicinal therapy even though the treatment process is simple since it is difficult to screen a proper drug that is free from side effects. For the medicinal therapy to be successful, the drug can regulate the imbalance of immune response, has to be safe, and has low relapse rate after a long-term administration. The most representative drugs used clinically nowadays are cyclosporine A and FK506, which are natural compounds but ask high costs and have been reported to cause various side effects accompanied by a long-term administration. Therefore, it is urgently requested to develop a novel immunosuppressant to overcome the above problems of the conventional drugs.

Metformin is an oral hypoglycemic agent that has long been used in the treatment of non-insulin dependent diabetes mellitus (NIDDM). It can lower the basic plasma glucose and after-meal plasma glucose, suggesting that metformin improves glucose tolerance in NIDDM patients (Zhou G, Myers R, Li Y, Chen Y, Shen X, Fenyk-Melody J, Wu M, Ventre J, Doebber T, Fujii N, Musi N, Hirshman M, Goodyear L, Moller D. Role of AMP-activated protein kinase in mechanism of metformin action. J Clin Invest. 2001; 108(8):1167-74). Metformin has been widely used as a type II diabetes treating drug. Recently, clinical approach has been made with metformin to treat polycystic ovary syndrome, weight loss, and cancer. However, there is no report saying that metformin can be used for the prevention or treatment of immune disease.

### DISCLOSURE

### Technical Problem

It is an object of the present invention to provide a composition comprising a metformin compound or a pharmaceutically acceptable salt thereof as an active ingredient for the prevention or treatment of immune disease induced by a pathogenic B cell, wherein the metformin is characterized by suppressing or reducing the activity of the pathogenic B cell.

### Technical Solution

To achieve the above object, the present invention provides a composition comprising a metformin compound or a pharmaceutically acceptable salt thereof as an active ingredient for the prevention or treatment of immune disease induced by a pathogenic B cell, wherein the metformin is characterized by suppressing or reducing the activity of the pathogenic B cell.

In a preferred embodiment of the present invention, the pathogenic B cell is CD138+B220− cell or GL7+B220+ cell.

In a preferred embodiment of the present invention, the suppression or reduction of the activity of B cell is attributed to the suppression or reduction of STAT3 activity; inducement or increase of AMPK activity; or inducement or increase of p53 activity.

In a preferred embodiment of the present invention, the metformin can suppress or reduce the activity of a pathogenic Th17, or accelerate or increase the activity of regulatory T cell (Treg).

In a preferred embodiment of the present invention, the suppression or reduction of Th17 activity or the acceleration or increase of regulatory T cell activity is attributed to the inducement or increase of AMPK activity; inducement or increase of Nrf2 activity; or inducement or increase of p53 activity.

In a preferred embodiment of the present invention, the immune disease is the disease caused by B cell, which can be selected from the group consisting of lupus, rheumatoid arthritis, psoriasis, inflammatory Bowel diseases, allergic rhinitis, asthma, renal fibrosis, carditis, B cell lymphoma, hypertension, tumor, and cancer.

In a preferred embodiment of the present invention, the preferable concentration of metformin is 1 μM˜100 μM.

In a preferred embodiment of the present invention, the metformin is to induce regulatory B cell activity.

In a preferred embodiment of the present invention, the regulatory B cell is IL-10+ B cell or Foxp3+ B cell.

The present invention also provides a method for reducing or suppressing in vitro differentiation of undifferentiated B cell into a pathogenic B cell comprising the step of treating metformin to the undifferentiated B cell.

In a preferred embodiment of the present invention, the metformin can suppress the differentiation of pro/pre B cell in the course of differentiation, CD138+B220− long lived plasma B cell, and GL7+B220+ germinal center B cell.

The present invention further provides a method for inducing in vitro activation of regulatory B cell comprising the step of treating metformin to the undifferentiated B cell.

In a preferred embodiment of the present invention, the regulatory B cell is IL-10+ B cell or Foxp3+ B cell.

In a preferred embodiment of the present invention, the activation of regulatory B cell is attributed to the metformin mediated suppression of STAT3 activity and the metformin mediated increase of AMPK activity and p53 activity.

The present invention provides a composition comprising a metformin compound or a pharmaceutically acceptable salt thereof as an active ingredient for the prevention or treatment of immune diseases, wherein the composition is characterized by suppressing or reducing the activity of a pathogenic B cell.

The present inventors paid attention to metformin as a novel treatment agent for immune diseases. Particularly, the present inventors first confirmed that metformin had the effect of preventing or treating immune disease by regulating the activity of a pathogenic B cell.

Up to date, metformin has been known to be effective mainly in treating type II diabetes. Korean Patent Publication No. 2009-005513 describes that metformin malonate has the anti-diabetic activity. However, there is no reports on the immune disease preventing or treating effect of metformin, yet.

In the studies on immune diseases, the major target has been T cell. Under the normal condition, immune system controls autoantigen specific immune response and often suppresses immune response against foreign antigen. This immune tolerance is attributed to clonal deletion, anergy, and Treg. In particular, immune regulatory T lymphocyte seems to be involved in transplantation immune response, autoimmunity, tumor immunity, and infection immune response.

The present inventors administered metformin to the lupus animal model. As a result, the levels of lupus-specific dsDNA and IgG were significantly decreased (see FIG. 1a), the size of the spleen of the lupus animal model administered with metformin was significantly reduced, and the size and the number of germinal center follicle (GC follicle) in the spleen were also significantly reduced. In addition, the infiltration of inflammatory cells around the blood vessels in the liver tissue was also reduced (see FIG. 1b). Based on the above results, the present inventors noticed that metformin could affect the activity of a pathogenic B cell. The increased B cell activity confirmed in the lupus animal model also supported the notification.

The role of B cell in autoimmune disease is further explained hereinafter. The autoreactive B cells not eliminated through central tolerance and peripheral tolerance in the course of B cell growth induce autoimmune disease via various mechanisms. The mechanism that causes autoimmune disease differs from the type of disease. For example, the autoreactive B cells can be converted into plasma cells to form autoantibodies, or form an immune complex by being accumulated in tissue to cause inflammatory response, or deliver autoantigen to T cells to activate autoantigen reactive T cells, or secrete inflammatory cytokines, or induce and progress autoimmune disease via the mechanism producing lymphocytic tissue in the abnormal area.

To confirm whether or not metformin was effective enough to induce treatment effect on disease by affecting the activity of a pathogenic B cell, the present inventors treated metformin to the lupus animal model group and three weeks later separated B cell from the animal group, followed by observation.

As a result, in the lupus animal model group treated with metformin, the representative pathogenic B cells, which were CD138+B220− long lived plasma B cells and GL7+B220+ germinal center B cells, were significantly reduced, compared with the control lupus animal model group that were not treated with metformin (see FIG. 2a). In the meantime, the spleen cells were extracted from the lupus mouse group treated with metformin, followed by staining thereof. As a result, the size of GC was significantly reduced, and also the number of follicular B helper T cells (THF) was also reduced (see FIG. 2b). B cell mediated IgG production was also reduced by metformin dose-dependently, confirmed by in vitro experiment (see FIG. 2c).

Based on the above results, the present inventors concluded that metformin was effective in treating lupus by suppressing or reducing the activity of a pathogenic B cell.

Further, the present inventors observed whether or not metformin could inhibit cell differentiation in the course of B cell differentiation, along with STAT3, AMPK, and p53, known as B cell activity regulators, in order to identify the mechanism that is responsible for the regulation of B cell activity. As a result, in the lupus animal model group treated with metformin, p-STAT3 705 and p-STAT3 727 were significantly down-regulated, while pAMPK and p53 were significantly up-regulated (see FIGS. 3a and 3b). Differentiations of pro/pre B cells in the stage of early differentiation and long lived plasma cells and germinal center B cells were all suppressed. Therefore, the present inventors could concluded that the mechanism responsible for the suppression or reduction of B cell activity was attributed to the suppression or reduction of STAT3 activity; inducement or increase of AMPK activity; or inducement or increase of p53 activity.

The present inventors further examined the possibility of metformin to regulate not only pathogenic B cell activity but also T cell activity based on the close interaction between T-cell and B-cell in immune response.

As a result, in the lupus animal model group treated with metformin, the pathogenic Th17 cell was reduced, but Treg was increased (see FIG. 4a). When metformin was treated to the pathogenic Th17 cells, AMPK expression was increased and also p-AMPK, nrf2 and p53 were up-regulated (see FIGS. 4b and 4c). Therefore, it was confirmed that the suppression or reduction of the pathogenic Th17 cell was attributed to the inducement or increase of AMPK activity; inducement or increase of Nrf2 activity; or inducement or increase of p53 activity.

The metformin of the present invention is also characterized by inducing regulatory B cell activity.

There are immune cells each playing a different role in human body. General immune cells are functioning to prevent the invasion of bacteria. On the other hand, regulatory immune cells are functioning to regulate balance of immune response to prevent excessive immune response. Those immune response regulating cells were known as T cells, but recently B lymphocytes were also confirmed to regulate immune response.

That is, the recent identified immune cell, regulatory B cell (Breg), is involved in generating immune substances and is also involved in the generation of Foxp3 protein that has been known to be expressed only in T lymphocytes so far. According to the recent studies, the lack of Breg or the inactivation or suppression of Breg activity can cause immune disease.

Therefore, it is expected to prevent and treat immune diseases resulted from abnormal immune response by activating regulatory B cell (Breg).

Thus, the present inventors investigated whether or not immune disease could be treated by the regulatory B cell activated by metformin. Particularly, in a preferred embodiment of the present invention, p53 which is one of the phenotypes of regulatory B cells was significantly increased in B cells treated with metformin, compared with the metformin non-treated B cells. Another phenotype of regulatory B cell, AMPK, was activated by metformin, while STAT3 activity was suppressed.

The present inventors further investigated whether or not the treatment of metformin could result in the promotion of the differentiation of undifferentiated B cell into regulatory B cell (Breg). Particularly, undifferentiated B cell was treated with metformin, followed by examination of IL-10+ B cell or Foxp3+ B cell. As a result, the numbers of both IL-10+ B cell and Foxp3+ B cell were all increased.

Therefore, the present invention was characterized by the first invention that was proved that the mechanism of metformin to treat immune disease is functioning to reduce the pathogenic Th17 cell but to increase regulatory T cell and to activate regulatory B cell to bring the treatment effect on immune disease.

The immune regulatory T cell, that is immune regulatory T lymphocyte (Treg), is divided largely into two groups, which are natural Treg ad adaptive Treg. CD4+CD25+ T cell, the natural Treg, is functioning to suppress immune response from the generation in thymus and takes 5˜10% of peripheral CD4+ T lymphocyte of a normal subject. The immunosuppressive mechanism of this cell has not been clearly understood yet, and only was confirmed the fact that Foxp3 functioning to suppress a certain gene expression plays an important role in the differentiation and activation of this cell. The peripheral natural T cell can be differentiated into a cell that has the effect of immune suppression when it receives stimulus from autoantigen or foreign antigen under a certain environment, which is called adaptive or inducible Treg. Tr1 secreting IL-10, Th3 secreting TGF-β, and CD8 Ts are included therein.

T cell can also be differentiated into Th17 cell via another differentiation process except Treg cell. Th17 cell differentiation is achieved in the presence of TGF-β, like Treg cell. However, Treg cell does not need IL-6 for its differentiation, while Th17 cell can only be differentiated in the presence of both TGF-β and IL-6. Th17 is characterized by the secretion of IL-17.

Th17 cells characteristically have cytotoxicity that can accelerate the progress of disease by maximizing the signal of inflammation response. So, the suppression of Th17 cell differentiation or activation can be a method for treating immune disease.

In this description, the regulatory B cell (Breg) is the B cell capable of regulating immune system similarly to the regulatory T cell (Treg), which secretes Foxp3 protein and IL-10.

To investigate how far metformin could affect the activation of regulatory B cell, the present inventors measured the levels of Foxp3 protein and IL-10 according to the treatment of metformin.

Foxp3 mainly exists in regulatory T cell originated from thymus and is functioning as a transcriptional factor working in the cell having CD4+CD25+ labeled antigens. Particularly, Foxp3 displays a low reactivity against the antigen when an antigen against T cell expressing Foxp3 is recognized and at the same time plays a role as a suppressor T cell that suppresses the IL-2 generation and cell division from those T cells that can induce autoimmune disease among CD4+CD25− T cells not expressing Foxp3 which are originated from thymus.

Foxp3 can also be found in regulatory B cell. Like regulatory T cell, regulatory B cell can suppress or regulate immune response so that they can be used for the treatment of immune disease. Regulatory T cell secretes the cytokine IL-10, and regulatory B cell (Treg) also secretes the cytokine IL-10.

Therefore, in this invention, the measurement of the activity and differentiation of regulatory B cell was performed by the same manner as the method to measure the generation of Foxp3 protein and IL-10 and to count the numbers of Foxp3+ and IL-10+ cells.

The regulatory B cell of the invention can be IL-10+ B cell or Foxp3+ B cell.

The present invention also provides a method for reducing or suppressing in vitro differentiation of undifferentiated B cell into pathogenic B cell comprising the step of treating metformin to the undifferentiated B cell.

The metformin characteristically suppresses the differentiation of pro/pre B cell in the course of B cell differentiation, CD138+B220− long lived plasma B cell, and GL7+B220+ germinal center B cell.

The present invention further provides a method for inducing in vitro activation of regulatory B cell comprising the step of treating metformin to the undifferentiated B cell.

The activation of regulatory B cell according to the present invention is achieved characteristically by the suppression of STAT3 activity by treating metformin thereto and by the promotion of AMPK activity and p53 activity.

STAT (signal transducers and activators of transcription) is a signal transduction and transcription regulatory protein, which is activated by such extracellular stimuli as cytokine, hormone, and growth factor. The activation of STAT results in the phosphorylation of tyrosine residue and the formation of dimer via interaction with SH2 domain. The dimer enters the nucleus and binds to a specific promoter therein. Such STAT protein signaling pathway can be suppressed by dephosphorylation and protein degradation.

The activated STAT1, STAT3, and STAT5 are identified in various cancers. In particular, STAT3, which is identified in not only blood cancer like leukemia but also solid tumor such as breast cancer, head/neck cancer, melanoma, ovarian cancer, lung cancer, pancreatic cancer, and prostate cancer, becomes an important target of anticancer treatment (Hua Yu and Richard Jove, Nature Review. Cancer, 2004, 4, 97-105.

The activation of STAT3 suppresses apoptosis, induces angiogenesis, and induces immune privilege (Wang T. et al., Nature Medicine., 2004, 10, 48-54). So, the suppression of STAT3 activity has the effect of tumor control through a combined anticancer mechanism. In addition, STAT3 protein is involved not only in tumor but also in various intracellular functions, suggesting that the screening of a STAT3 activity inhibitor leads to the development of an immunosuppressant.

Based on the above description, the composition comprising a metformin compound or a pharmaceutically acceptable salt thereof as an active ingredient can be used for the prevention or treatment of B cell related immune diseases because of its activity of suppressing or reducing the activity of a pathogenic B cell. The disease can be lupus, rheumatoid arthritis, psoriasis, inflammatory Bowel diseases, allergic rhinitis, asthma, renal fibrosis, carditis, B cell lymphoma, hypertension, tumor, or cancer, but not always limited thereto.

The metformin compound, the pharmacologically active ingredient of the invention, can be the compound represented by formula 1.

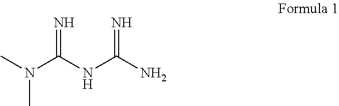

The compound represented by formula 1 of the present invention can be used in the form of a pharmaceutically acceptable salt. As for the pharmaceutically acceptable salt, it is preferably an acid addition salt prepared by use of a pharmaceutically acceptable free acid. Whether it is inorganic or organic, a free acid can be used if it is pharmaceutically acceptable. Available organic free acids are exemplified by citric acid, acetic acid, lactic acid, malic acid, maleic acid, fumaric acid, formic acid, propionic acid, oxalic acid, trifluoroacetic acid, benzoic acid, gluconic acid, methanesulfonic acid, gluconic acid, succinic acid, 4-toluenesulfonic acid, glutamic acid, and aspartic acid, but not always limited thereto. Examples of the inorganic free acid include hydrochloric acid, bromic acid, nitric acid, and phosphoric acid, but not always limited thereto.

The compound of the present invention can be isolated from natural sources or synthesized by a chemical synthesis method well known to those in the art.

The immune disease herein indicates a pathologic condition that is caused, mediated, or triggered by the components of a mammal immune system by any means. When immune response is stimulated or terminated, which means any abnormal immune response occurs, this situation affects the progress of disease, that is it brings compensation effect. This case is also included in the criteria of the immune disease of the present invention. For example, the diseases caused by hypersensitive immune response are all included in this invention. Thus, the immune disease of the present invention is exemplified by autoimmune disease; inflammatory disease; and cell, tissue, or organ transplantation rejection related disease, but not always limited thereto.

One of the most important immune related functions of a normal subject is not to respond negatively to self antigens but to recognize, respond, and eliminate non-self antigens. Such unresponsiveness of a living body against autoantigen is called immunologic unresponsiveness or tolerance.

When there is a problem in inducing or keeping such self tolerance, immune response even against autoantigen is induced, resulting in attacking self tissue. The disease developed by such progress is called autoimmune disease.

The term “treatment” in this invention, indicates the action that can reverse or relieve the disease itself or one or more symptoms of the disease targeted by the term of the invention, or that can inhibit the progress or prevent the target disease. Therefore, the treatment or therapy for immune disease in mammals can contain at least one of the followings:

(1) inhibiting the growth of immune disease, that is the inhibition of the development,

(2) preventing the spread of immune disease, that is the prevention of transference,

(3) alleviating immune disease.

(4) preventing recurrence of immune disease, and

(5) palliating the symptoms of immune disease.

The composition of the present invention for the prevention or treatment of immune disease can include a pharmaceutically effective dose of the compound represented by formula 1 or the pharmaceutically effective salt thereof alone or together with one or more pharmaceutically acceptable carriers, excipients, or diluents. The pharmaceutically effective dose herein indicates the amount enough to prevent, improve, and treat the symptoms of immune disease.

The pharmaceutically effective dose of the metformin compound of the present invention or the slat thereof is 0.5˜100 mg/day/weight kg, preferably 0.5˜5 mg/day/weight kg. However, the pharmaceutically effective dose above can be adjusted according to the severity of symptoms of immune disease, age, weight, health condition, and gender of patient, administration pathway, and treatment period, etc.

The “pharmaceutically acceptable” herein indicates a composition that is acceptable physiologically and does not cause any allergic reaction or allergy like reaction such as gastrointestinal disorder and dizziness. The carriers, excipients and diluents are exemplified by lactose, dextrose, sucrose, sorbitol, mannitol, xylitol, erythritol, maltitol, starch, acacia rubber, alginate, gelatin, calcium phosphate, calcium silcate, cellulose, methyl cellulose, microcrystalline cellulose, polyvinyl pyrrolidone, water, methylhydroxybenzoate, propylhydroxybenzoate, talc, magnesium stearate and mineral oil. In addition, fillers, antiaggregations, lubricants, wetting agents, flavors, emulsifiers, and antiseptics, can also be included.

The composition of the present invention can be formulated properly in a proper form by those in the art, in order for the active ingredient to be delivered fast, continuously, or delayed purposefully after administered to mammals. The composition can be formulated in the forms of powders, granules, tablets, emulsions, syrups, aerosols, soft or hard gelatin capsules, sterilized injections, or sterilized powders.

The pharmaceutical composition of the present invention for the prevention or treatment of immune disease can be administered via various pathways including oral administration, transdermal, hypodermic, intravenous, and intramuscular injection. The dose of the active ingredient can be adjusted according to the administration pathway, age, gender, and weight of patient, severity of disease, etc. The composition for the prevention or treatment of immune disease of the invention can be co-administered with any informed compound that is known to have the effect of preventing, improving, or treating immune disease.

The present invention thus provides a pharmaceutical composition comprising a metformin compound or a salt thereof as an active ingredient for the prevention or treatment of immune disease and further the invention provides a composition for immunosuppression comprising a metformin compound or a salt thereof as an active ingredient.

The present invention also provides a food composition comprising a metformin compound or a salt thereof as an active ingredient for the improvement or prevention of immune disease. The food composition of the present invention can be used as the functional food for improving or preventing the symptoms of immune disease, which can be applied as a main material for food, a sub-material for food, or a food additive, or applied to the functional food or beverages.

The term “food” in this invention indicates a natural or processed product containing one or more nutrients, and more preferably an edible product finished with food process. In general, food, food additives, and functional food and beverages are all included.

Food to which the food composition of the present invention can be added is exemplified by beverages, gums, tea, vitamin complex, other functional food, etc. Particularly, the applicable food for the present invention includes special nutritious food (ex: milk formulas, baby food, etc), processed meats, processed fish products, tofu, muk, noodles (ex: ramyen, noodle, etc), bread, health supplement food, seasoning food (ex: soy sauce, soybean paste, red pepper paste, mixed soy paste, etc), sauces, confectionery (ex: snacks), candies, chocolates, gums, ice creams, milk products (ex: fermented milk, cheese, etc), other processed food, kimchi, pickles (a variety of kimchi, pickled vegetables, etc), beverages (ex: fruit juices, vegetable juices, soy drinks, fermented drinks, etc), and natural spices (including ramyen soup), but not always limited thereto. These food, beverages, or food additives can be prepared by the conventional method for food product.

The “functional food” herein is the food group to which a specific value is added by using physical, biochemical, and biotechnological techniques in order to fit or express a purposed function, or the processed food designed to express the biological regulation function such as regulating bio-rhythm, preventing disease or helping recovery from disease, more specifically indicates the health improving functional food. The functional food can contain any acceptable food additive, precisely any general carrier, excipient, and diluent that have been generally added to the functional food.

The “beverage” in this invention is the general term for anything to drink to quench or enjoy tastes, which includes functional beverages. Ingredients for such beverages are not limited except that the active ingredient, the composition for the improvement or prevention of immune disease, has to be included. This functional beverage can additionally include various flavors or natural carbohydrates like any other beverages do.

In addition to the ingredients mentioned above, the food comprising the food composition for the improvement or prevention of immune disease of the present invention can include in variety of nutrients, vitamins, minerals (electrolytes), flavors including natural flavors and synthetic flavors, coloring agents and extenders (cheese, chocolate, etc.), pectic acid and its salts, alginic acid and its salts, organic acid, protective colloidal viscosifiers, pH regulators, stabilizers, antiseptics, glycerin, alcohols, carbonators which used to be added to soda, etc. All the mentioned ingredients can be added singly or together.

The concentration of the food composition of the present invention in the applicable food is 0.001 weight %˜90 weight % by the total weight of the food, more preferably 0.1 weight %˜40 weight %. For the beverages, the concentration of the food composition of the present invention in 100 ml of beverages is 0.001 g˜2 g, and more preferably 0.01 g˜0.1 g. However, if long term administration is required for health and hygiene or for regulating health condition, the content can be lower than the above but higher content can be accepted as well since the active ingredient has been proved to be very safe.

In this invention, “activity” or “activation” indicates that all the mechanisms of cells or molecules are accelerated or increased.

### Advantageous Effect

The present invention provides a composition comprising a metformin compound that is effective in treating immune diseases caused by abnormal immune response as an active ingredient for the prevention or treatment of immune diseases.

The present invention also has the effect of providing the mechanism of the metformin compound that is used as a composition for the prevention or treatment of immune diseases.

The present invention further provides a use of the metformin compound for immune diseases with presenting the possible preventive or treatable immune diseases.

### DESCRIPTION OF DRAWINGS

The application of the preferred embodiments of the present invention is best understood with reference to the accompanying drawings, wherein:

FIG. 1a is a graph illustrating the down-regulation of lupus-specific dsDNA and IgG by the treatment of metformin in the lupus animal model group treated with metformin.

FIG. 1b illustrates the changes of the size of spleen and other changes in spleen and liver tissues by the treatment of metformin in the lupus animal model group treated with metformin, observed under optical microscope.

FIG. 2a illustrates the significant decrease of a pathogenic B cell which in the lupus animal model group treated with metformin.

FIG. 2b illustrates the changes of germinal center (GC) and Tfh in the spleen of the lupus animal model group treated with metformin.

FIG. 2c illustrates the results of in vivo experiment saying that the IgG production by the pathogenic B cell is reduced by metformin dose-dependently.

FIG. 3a illustrates the results of another experiment saying that pSTAT 705 and p-STAT 727 are reduced in B cells of the lupus animal model group treated with metformin.

FIG. 3b illustrates that pAMPK and p53 are increased in B cells of the lupus animal model group treated with metformin.

FIG. 3c illustrates that the activity and the number of the cells in the course of B cell differentiation are suppressed when the B cells that have been activated by LPS in vitro are treated with metformin.

FIG. 4a illustrates that regulatory T cells are increased but pathogenic Th17 cells are decreased in the lupus animal model group treated with metformin.

FIG. 4b illustrates the up-regulation of AMPK and p-AMPK in the lupus animal model group treated with metformin.

FIG. 4c illustrates the increase of Nrf2 and p53 in the lupus animal model group treated with metformin.

FIG. 5 illustrates the results of an experiment showing the regulation of T_{FH} cell activity by metformin in the autoimmune disease animal model.

FIG. 6a˜FIG. 6c illustrate the results of an experiment showing the regulation of Th17 cell activity by metformin in the autoimmune disease animal model.

FIG. 7a˜FIG. 7d illustrate the results of an experiment showing the suppression of mTOR/STAT3 activity by metformin in the autoimmune disease animal model.

FIG. 8a˜FIG. 8b illustrate the regulation of autophagy activity by metformin in the autoimmune disease animal model.

FIG. 9a˜FIG. 9c illustrate the suppression of Th17 cell activity mediated by the regulation of autophagy activity by metformin in the autoimmune disease animal model.

### BEST MODE

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Disease Relieving Effect of Metformin in the Lupus Animal Model

To investigate the treatment effect of metformin on lupus, one of the immune diseases, the present inventors administered metformin to the lupus animal model orally at the dose of 100 mg/kg every day for 3 weeks. At this time, the lupus animal model not treated with metformin was used as the control. Also, the normal mouse group that did not have the disease was prepared for the comparison (wild type). The analysis of the treatment effect was performed as follows. Blood was extracted from each experimental group 1 week after the metformin administration. Serum was separated from the blood extracted above. The levels of lupus-specific dsDNA (Sigma, cat no. D8515) and IgG (Sigma) in the separated serum were measured by ELISA. Particularly, 96-well plate was coated with dsDNA or anti-mouse IgG, to which the serum separated from the mouse blood was loaded, followed by reaction. Then, anti-HRP antibody was added thereto, followed by reaction. Color development was induced by TMB system and the fluorescence was measured.

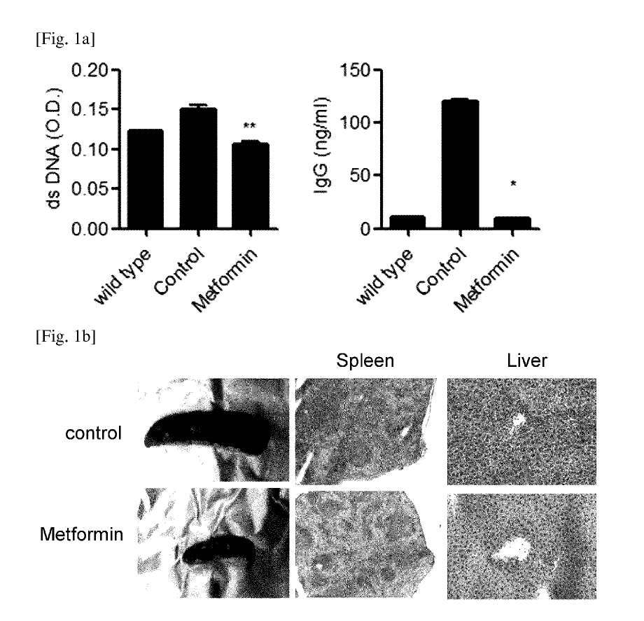

As a result, the levels of lupus-specific dsDNA and IgG were significantly reduced in the mouse model group treated with metformin, unlike the group not treated with metformin. The level of lupus-specific dsDNA in the group treated with metformin was rather lower than that of the normal mouse group. The level of IgG in the lupus mouse model treated with metformin was similar to that of the normal mouse group (FIG. 1a).

Spleen and liver were extracted from the lupus mouse model treated with metformin and from the control group that was not treated with metformin. The size of those spleen and liver was measured by the naked eye, and the size and the number of GC follicle (Germinal Center follicle) were also observed.

As a result, the size of spleen was significantly reduced in the lupus mouse model treated with metformin, compared with that of the group not treated with metformin. The size and the number of GC follicle were also significantly decreased by the treatment of metformin (FIG. 1b).

The infiltration of inflammatory cells around the blood vessels of liver tissue was also observed. As a result, the infiltration of inflammatory cells around the blood vessels of liver tissue in the lupus mouse group treated with metformin was hardly observe, while the infiltration of inflammatory cells was clearly observed in the lupus mouse group not treated with metformin (FIG. 1b).

From the above results, it was confirmed that metformin had the effect of relieving lupus disease.

### Example 2

### Suppression of the Pathogenic B Cell Activity by Metformin

The lupus mouse model showing the increased B cell activity was administered with metformin orally at the dose of 100 mg/kg every day for 3 weeks. Then, spleen cells were separated therefrom and B cell was observed. Particularly, the representative pathogenic B cells, B220−CD138+ long lived plasma B cell and GL7+B220+ germinal center B cell, were observed. For the observation, the spleen cells separated from each group were stained with CD138 and B220 and the long lived plasma cells were stained with GL7 and B220, followed by FACS to examine the expression of each cell.

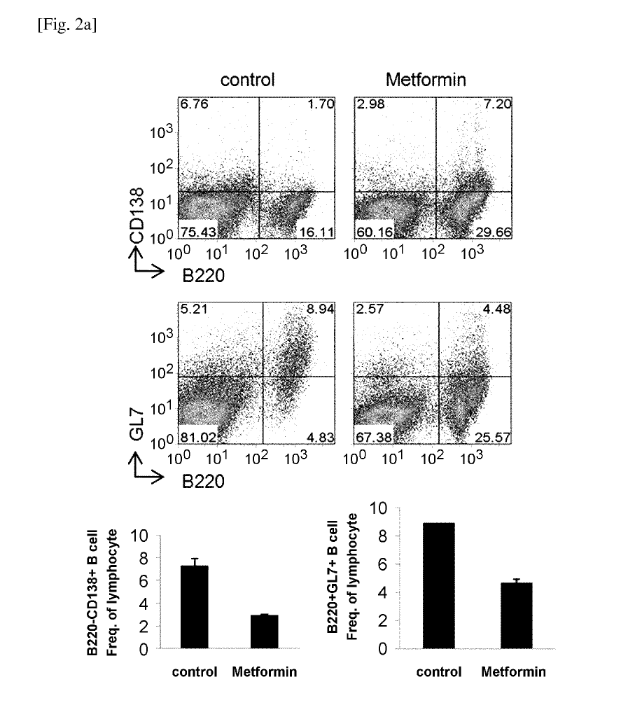

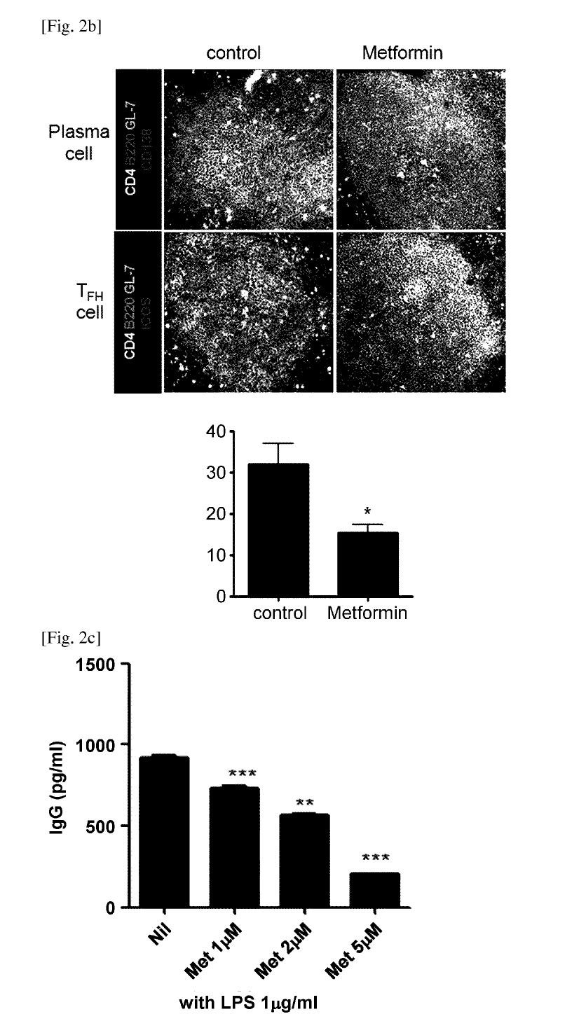

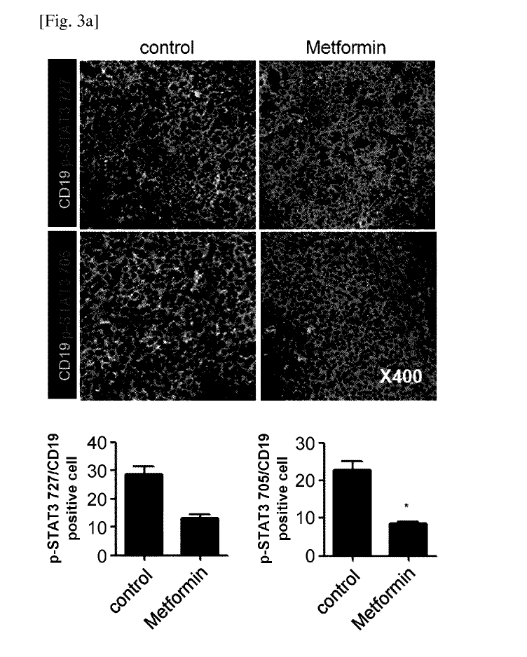

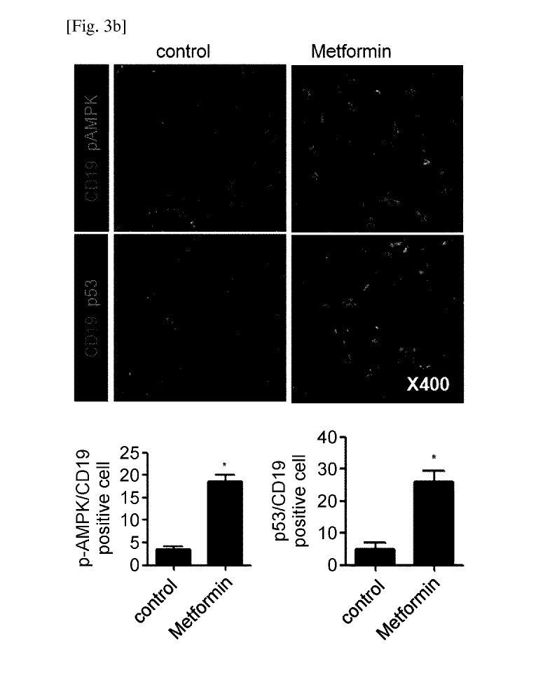

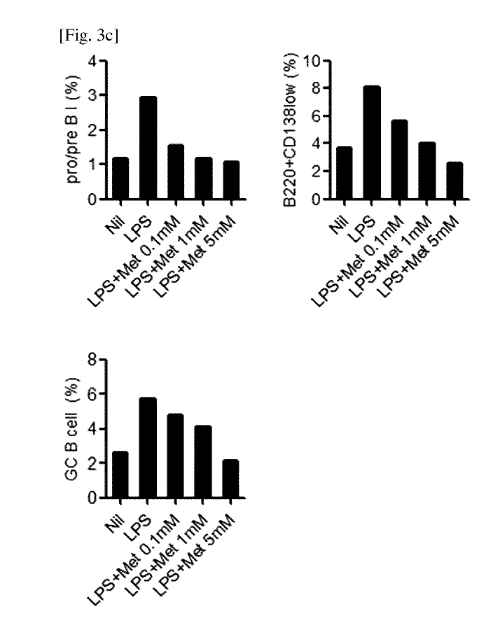

As a result, the pathogenic B cells, B220−CD138+ long lived plasma B cell and GL7+B220+ germinal center B cell, were significantly reduced in the lupus mouse model treated with metformin, compared with the control group (FIG. 2a). From the staining of spleen cells, it was also confirmed that the size of GC was significantly reduced in the lupus mouse model administered with metformin, compared with the lupus mouse model not treated with metformin, and the number of follicular B helper T cells (THF) was also reduced (see FIG. 2b).

To investigate whether or not metformin could inhibit the antibody production of a pathogenic B cell, the lupus mouse model was administered with metformin to at different concentrations (1 μM, 2 μM, and 5 μM), and stimulated with LPS (1 μg/ml). Then, the level of IgG produced by B cell of the mouse was measured by the same manner as described in Example 1.

As a result, as shown in FIG. 2c, the IgG production in B cell was reduced by metformin dose-dependently.

Therefore, it was confirmed that metformin could be effectively used for the prevention and treatment of immune disease caused by B cell by suppressing or reducing the activity of a pathogenic B cell.

### Example 3

### Regulation of STAT3 Suppression and Regulation of AMPK Activity and p53 Activity by Metformin

STAT3, AMPK, and p53 are all known as the molecules capable of regulating the pathogenic B cell activity, so the regulation of those factors under the situation of excessive immune response is very important. To investigate whether or not metformin mediated B cell activity suppression was attributed to the regulation of STAT3, AMPK, and p53, the present inventors observed the activities of those factors in the lupus mouse model group treated with metformin and of the control group not treated with metformin. Particularly, joints were extracted from the control group and the metformin treated experimental group, which were fixed in 10% neutral buffered formalin, followed by decalcification with EDTA. The decalcified joint was embedded in paraffin, which was sliced into 7 μM thick sections. The sections were placed on the slide. The slide was deparaffinized with xylene and hydrated with ethanol from high concentration to low concentration. The slide was stained with hematoxylin-eosin, followed by immunohistochemical staining. The stained p-STAT3 705, p-STAT3 727, p-AMPK, p53, and CD19 were observed under optical microscope.

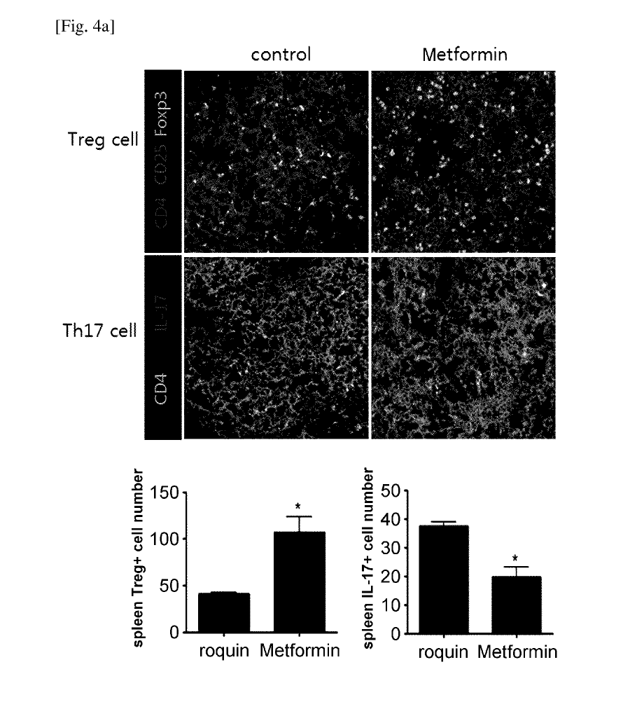

As a result, the levels of p-STAT3 705 and p-STAT3 727 were significantly reduced in B cell of the lupus animal model treated with metformin, compared with those of the control group. In the meantime, the level of p-AMPK was increased in B cell of the lupus animal model treated with metformin, compared with that of the control group. Particularly, the level of p53, one of the regulatory B cell phenotypes, was significantly increased in B cell of the lupus animal model treated with metformin (FIGS. 3a and 3b).

To investigate whether or not the cell activity could be regulated by metformin in each step of B cell differentiation, the present inventors treated the cell with LPS and metformin for 3 days. FACS was performed to observe any changes of the cell in each stage of B cell differentiation.

As a result, pro/preB cell in the stage of early B cell differentiation, pre-long lived plasma cell B220+CD138 low cell, and germinal center B cell were all suppressed by metformin dose-dependently (FIG. 3c).

The present inventors confirmed from the above results that metformin had the effect of treating immune diseases caused by the pathogenic B cell by suppressing STAT3 activity; inducing the activity of AMPK or p53 which is one of the regulatory B cell phenotypes; and suppressing or reducing the activity of a pathogenic B cell.

### Example 4

### Regulation of Regulatory T Cell (Treg) and Th17 Cell and Regulation of AMPK, Nrf2, and p53 Activity by Metformin

To investigate the effect of metformin to regulate Treg and the pathogenic Th17 cell, the present inventors observed the activity of regulatory T cell and Th17 cell in the lupus animal model group administered with metformin and the control group.

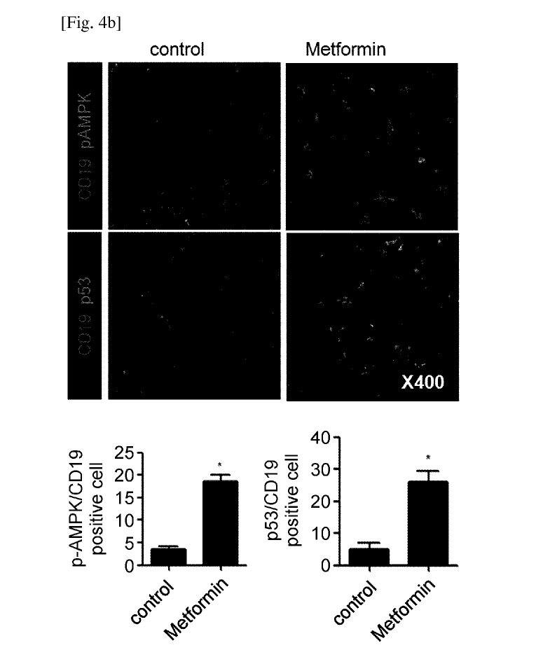

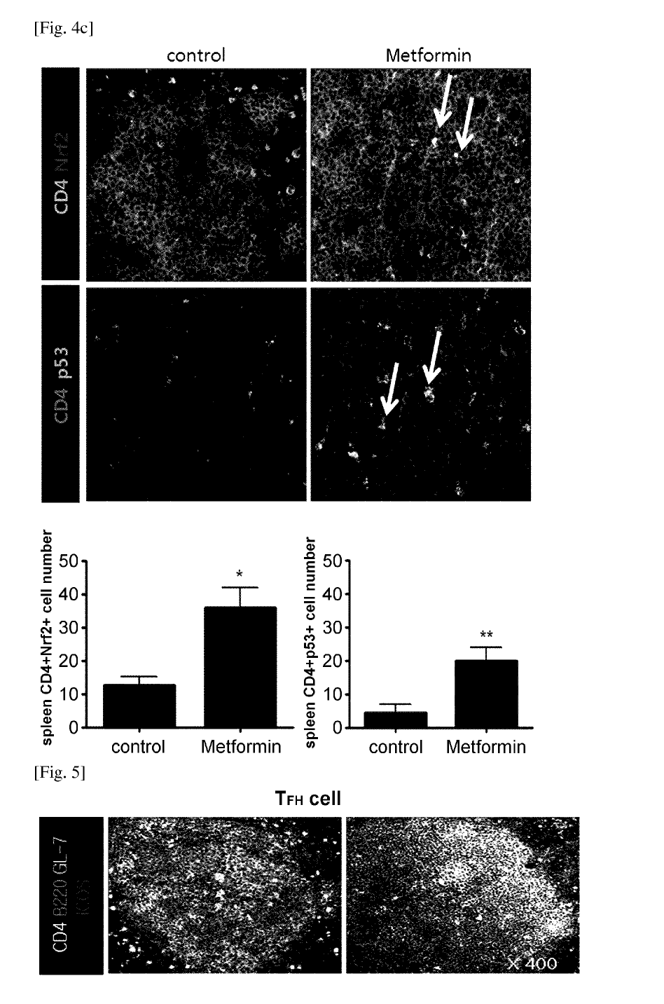

As a result, the activity of regulatory T cell was increased in the metformin treated lupus animal model group, while the activity of Th17 was decreased therein (FIG. 4a).

In the meantime, the expression and activity of AMPK was significantly increased in the metformin treated lupus animal model group, and so were the expressions of Nrf2 and p53 (FIGS. 4b and 4c).

The present inventors confirmed from the above results that the metformin of the present invention had the effect of preventing and treating immune diseases by suppressing not only B cell activity but also Th17 cell activity but at the same time by inducing regulatory T cell activity.

### Example 5

### Regulation of T_{FH} Cell Activity by Metformin in the Autoimmune Disease Animal Model

The lupus animal model was administered with metformin via intraperitoneal injection in the intervals of 2 days, 3 times a week. 5 weeks after the administration, T_{FH} cell expression in the spleen tissue of each mouse administered with metformin and of each mouse of control was investigated after performing confocal staining. Particularly, CD4-PerCP, B220-APC, GL-7-FITC, and IOCS-PE fluorescence staining was performed. Then, TFH cells expressing GL-7+ICOS+ in CD4 T cells were examined under fluorescent microscope.

As a result, the expression of T_{FH} cell expressing ICOS in the spleen tissue of the lupus animal model treated with metformin was suppressed (FIG. 5). In the lupus animal model, T_{FH} cell expressing ICOS in the spleen was over-expressed, by which GC formation and autoantibody production were accelerated. Thus, it was confirmed from this examination that metformin could control the activity of T_{FH} cell.

### Example 6

### Regulation of Th17 Cell by Metformin in the Autoimmune Disease Animal Model

The present inventors investigated whether or not metformin could suppress the expression of IL-17 with spleen cells of the lupus animal model. Particularly, T cells were separated from the spleen of the lupus mouse model by using CD4+ microbeads. The cells were treated with metformin under the condition of Th17 (ant-CD3 0.5 μg/ml, anti-CD28 1 μgmld, anti-IFNr 2 μg/ml, anti-IL-4 2 μg/ml, TGF-b 2 ng/ml, IL-6 20 ng/ml) cell differentiation (0.1, 1, and 5 mM) in vitro, followed by culture for 3 days. Upon completion of the culture, some of the cultured cells were stained with CD4-PerCP and ILo-17 PE, followed by FACS. RNA was separate from the remaining cells, from which Th17 cell related gene was amplified by real-time PCR. The expression of IL-17 in the culture solution was analyzed by ELISA (antigen-antibody examination).

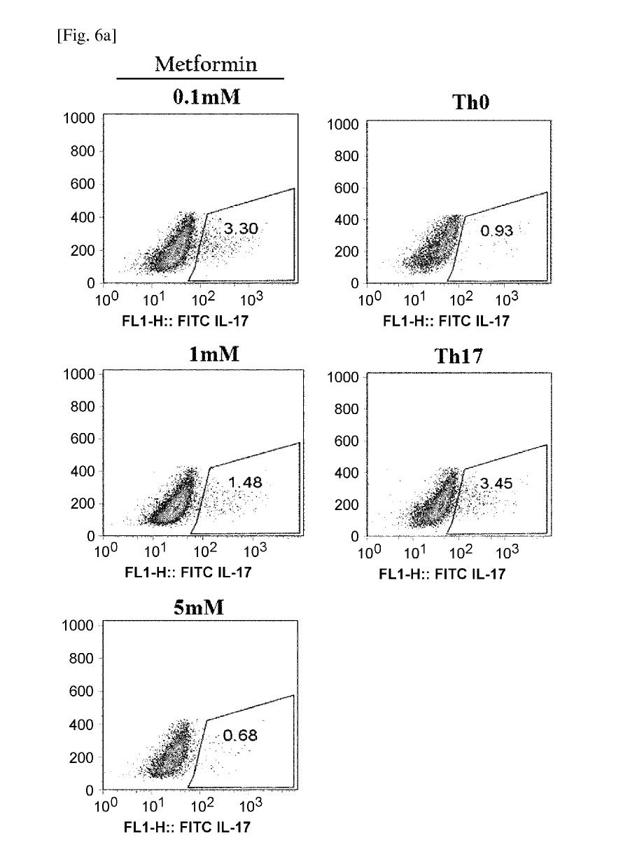

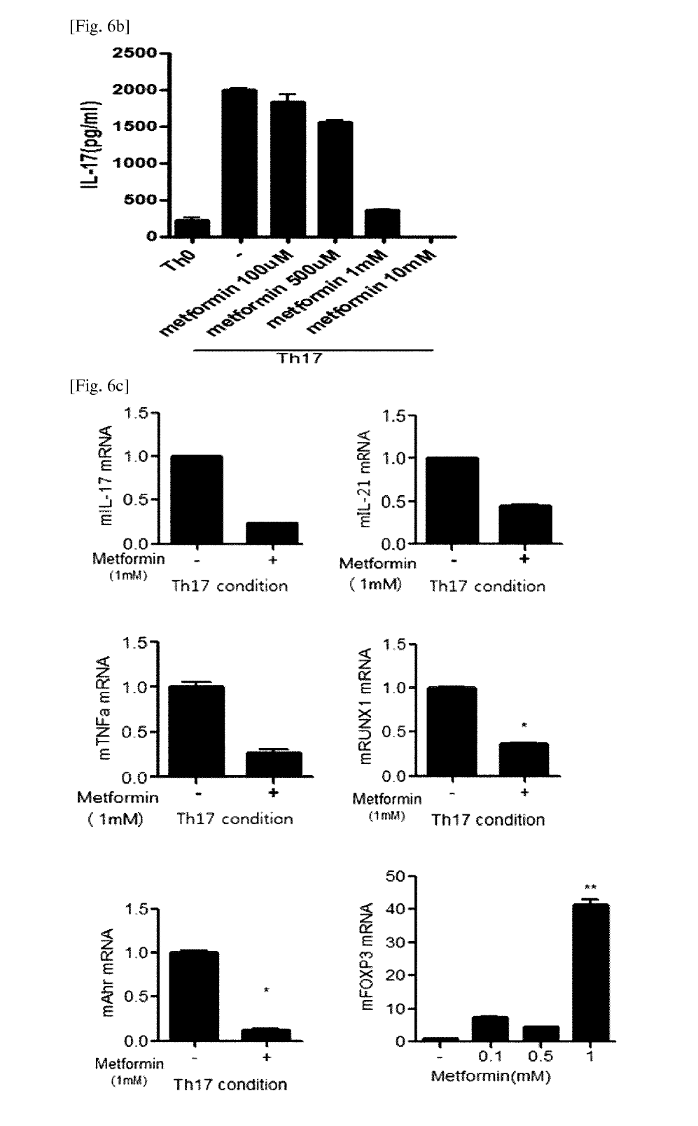

As a result, the expression of IL-17 in the lupus animal model was regulated by metformin (FIGS. 6a and 6b). Metformin was also involved in the suppression of the expressions of Th-17 related genes, IL-17, IL-21, TNF-α, RUNX1, and Ahr. It was additionally confirmed that metformin increased the expression of Foxp3 gene (FIG. 6c).

### Example 7

### Regulation of the Suppression of mTOR/STAT3 by Metformin in the Autoimmune Disease Animal Model

The present inventors investigated whether or not metformin could regulate mTOR/STAT3 with spleen cells of the lupus animal model at the cell level. Particularly, T cells were separated from the spleen cells of the lupus animal model. The separated T-cells were treated with IL-6 (10 ng/ml) or IL-2 (10 ng/ml) along with 1 mM of metformin, followed by culture for 24 hours. To analyze signal molecules in the cultured cells, protein was extracted from each condition of the cells. Western blotting was performed with the extracted protein. The protein separated by size was conjugated with each antibody against p-AMPK, AMPK, p-mTOR, mTOR, p-STAT3 705, p-STAST3 727, STAT3, p-STAT5, STAT5, and β-actin. The activity of each molecule was evaluated. Some of the cultured cells were conjugated with p-AMPK, AMPK, p-mTOR, p-STAT3 705, p-STAT3 727, and p-p53 fluorescent antibodies, whose expressions were examined under fluorescent microscope after confocal staining

In addition, nucleus and cytoplasm were separated in order to examine the expression of NrF2 in the nucleus and cytoplasm. Electrophoresis was performed and then Nrf2, tubulin, and β-actin were analyzed by Western blotting.

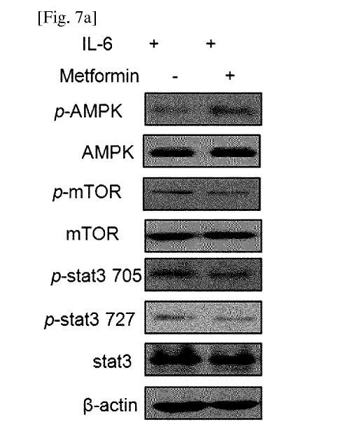

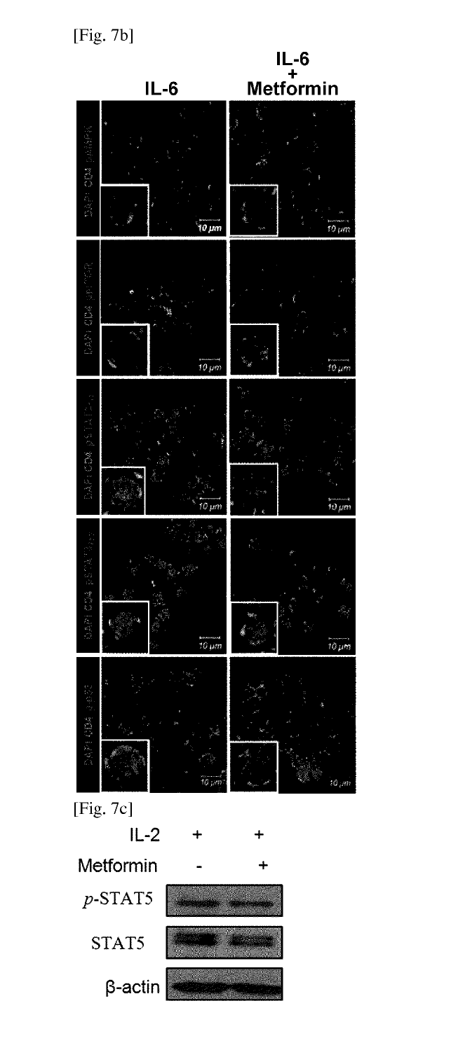

As a result, metformin activated AMPK and was thereby involved in the regulation of the suppression of mTOR/STAT3, and at the same time increased the expression of NrF2 that was a STAT3 suppression regulator and had the anti-oxidative activity (FIG. 7a˜FIG. 7d).

### Example 8

### Regulation of Autophagy by Metformin in the Autoimmune Disease Animal Model

The present inventors investigated whether or not metformin could activate autophagy with spleen cells of the lupus animal model. Particularly, T cells were separated from the spleen cells of the lupus animal model. The separated T cells were treated with 1 mM of metformin under the condition of Th17 cell differentiation, followed by culture for 24 hours. To analyze signal molecules in the cultured cells, protein was extracted from each condition of the cells. Western blotting was performed with the extracted protein. The protein separated by size was conjugated with each antibody against ATG5, p62, and 3-actin. The activity of each molecule was evaluated. Some of the cultured cells were conjugated with DAPI, IL-17, and Foxp3 fluorescent antibodies, whose expressions were examined under fluorescent microscope after confocal staining.

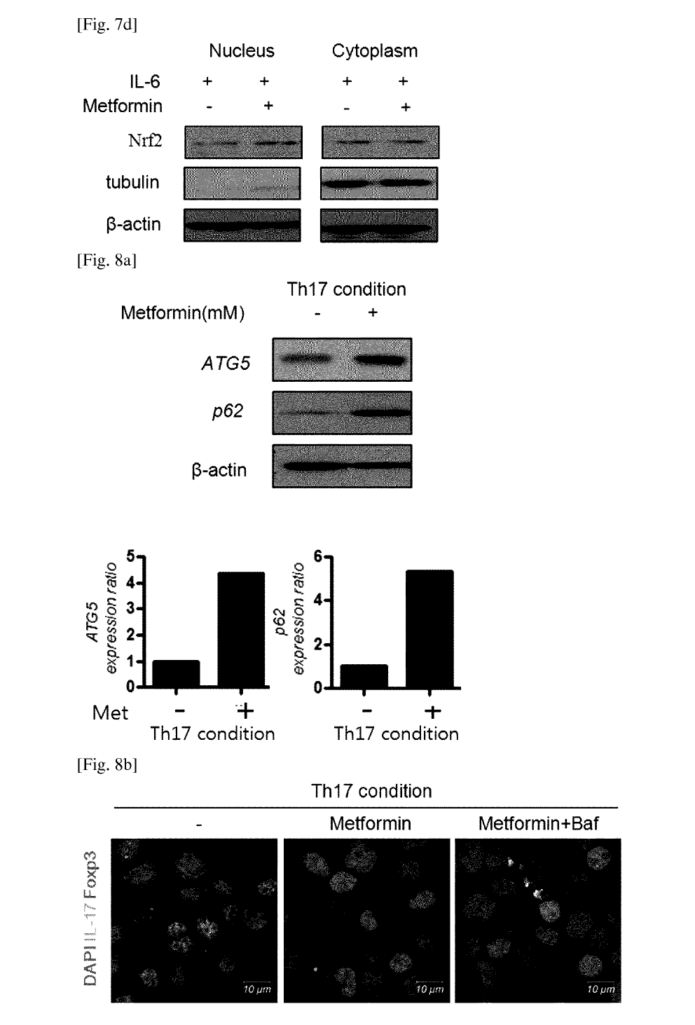

As a result, metformin was confirmed to activate ATG5 and p62, the autophagy activating molecules (FIG. 8a) and at the same time increase the expression of Foxp3 but suppress the expression of IL-17 (FIG. 8b). Therefore, metformin was confirmed to be able to regulate the activity of Th17/Treg cell by regulating the activity of autophagy.

### Example 9

### Suppression of Th17 According to the Regulation of Autophagy Activity by Metformin in the Autoimmune Disease Animal Model

Metformin was confirmed to be able to activate autophagy in the spleen cells of the lupus animal model in Example 8. Thus, the present inventors investigated whether or not the activation of autophagy could be directly involved in the suppression of Th17 cell by using an autophagy activity inhibitor. Particularly, T cells were separated from the spleen cells of the lupus mouse model. The separated T cells were treated with 1 mM of metformin or 10 μM of bafilomycin or methyladenine, the autophagy activity inhibitor, under the condition of Th17 cell differentiation, followed by culture for 72 hours. Upon completion of the culture, IL-17, IL-21, and TNF-α cytokines in the culture solution were analyzed by ELISA. Some of those cultured cells were observed under electron microscope to directly observe the activated autophagy therein.

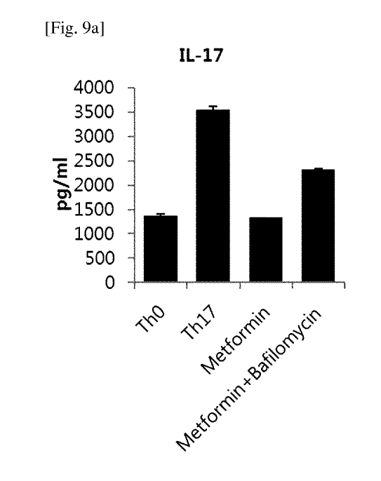

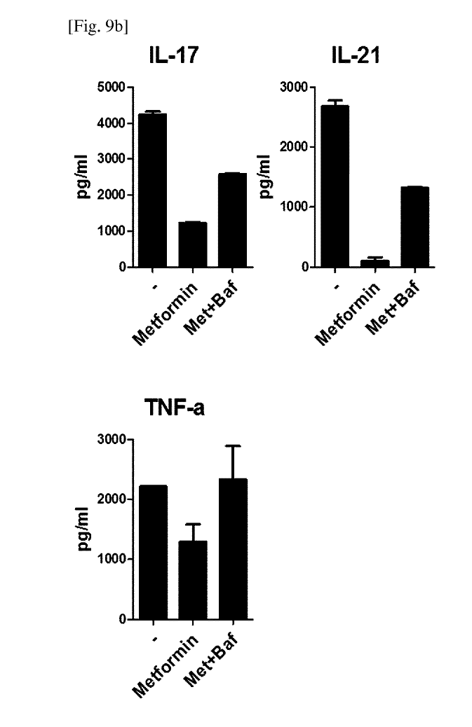

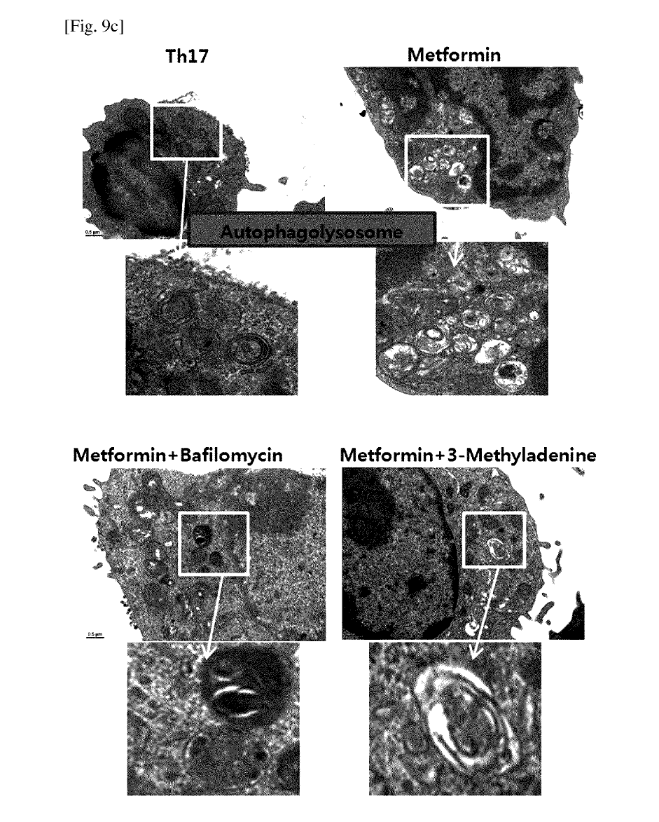

As a result, the expression of IL-17 was suppressed by the administration of metformin (FIG. 9a). At this time, the suppressed IL-17 expression was increased when the autophagy inhibitor was co-treated thereto (FIG. 9b). The intracellular activation of autophagy by metformin was directly confirmed by electron microscope (FIG. 9c). Therefore, it was confirmed that the activation of autophagy by metformin was directly related to the regulation of Th17 cell activity.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A composition comprising a metformin compound represented by the following formula 1 or a pharmaceutically acceptable salt thereof as an active ingredient for the prevention or treatment of immune disease induced by a pathogenic B cell.

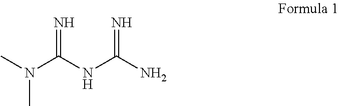

2. The composition according to claim 1, wherein the metformin is characterized by suppressing or reducing the activity of the pathogenic B cell.

3. The composition according to claim 1, wherein the pathogenic B cell is CD138+B220− cell or GL7+B220+ cell.

4. The composition according to claim 2, wherein the suppression or reduction of the activity of the pathogenic B cell is attributed to the mechanism selected from the group consisting of suppression or reduction of the STAT3 activity; induction or increase of the AMPK activity; and inducion or increase of the p53 activity.

5. The composition according to claim 1, wherein the metformin is characterized by suppressing or reducing the activity of a pathogenic Th17 cell, or by promoting or increasing the activity of a regulatory T cell.

6. The composition according to claim 5, wherein the suppression or reduction of the activity of the pathogenic Th17 cell or the promotion or increase of the activity of the regulatory T cell is attributed to the mechanism selected from the group consisting of inducion or increase of AMPK activity; inducion or increase of Nrf2 activity; and inducion or increase of p53 activity.

7. The composition according to claim 1, wherein the immune disease is selected from the group consisting of lupus, rheumatoid arthritis, psoriasis, inflammatory Bowel diseases, allergic rhinitis, asthma, renal fibrosis, carditis, B cell lymphoma, hypertension, tumor, and cancer.

8. The composition according to claim 1, wherein the metformin is included in the composition at the concentration of 1 μM˜100 μM.

9. The composition according to claim 1, wherein the metformin is characterized by inducing the activity of a regulatory B cell.

10. The composition according to claim 9, wherein the regulatory B cell is IL-10+ B cell or Foxp3+ B cell.

11. A method for reducing or suppressing in vitro differentiation of a undifferentiated B cell into a pathogenic B cell comprising the step of treating metformin to the undifferentiated B cell.

12. The method according to claim 11, wherein the metformin is characterized by suppressing the differentiations of pro/pre B cell in the stage of B cell differentiation, CD138+B220− long lived plasma cell, and GL7+B220+ germinal center cell.

13. A method for inducing in vitro activation of a regulatory B cell comprising the step of treating metformin to a undifferentiated B cell.

14. The method according to claim 13, wherein the regulatory B cell is IL-10+ B cell or Foxp3+ B cell.

15. The method according to claim 13, wherein the activation of the regulatory B cell is attributed to the metformin mediated suppression of STAT3 activity and the metformin mediated increase of AMPK and p53 activities.
